(19) United States
(12) Patent Application Publication
(10) Pub. No.: US 2022/0395409 A1
(43) Pub. Date: Dec. 15, 2022

(54) Belt Suspended Fully Detachable Cloth Diaper with Compatible Belted Garments

(71) Applicant: Chelsea Marian McAmis, Chattanooga, TN (US)

(72) Inventor: Chelsea Marian McAmis, Chattanooga, TN (US)

(21) Appl. No.: 17/346,892

(22) Filed: Jun. 14, 2021

(51) Int. Cl. A61F 13/56

(52) U.S. Cl. CPC A61F 13/5638

(57) ABSTRACT

An absorbent article and compatible garments for use on infants, children, or incontinent individuals. The absorbent article comprises a front part (21), rear part (22), intermediate crotch part (24), and front and rear fastening devices (30A-30B, 28A-28D). The compatible garments comprise a waist belt with front and rear fastening devices (44A-44B, 46A-46D) and features such as sleeves for legs (62A-62B) or a skirt (72), such that when such a garment is placed on a wearer, the wearer's groin and buttocks are uncovered. The absorbent article is fastened to the waist belt of the garment to cover the wearer's groin and buttocks. Thus, the absorbent article can be quickly attached to, detached from, partially attached to, or partially detached from the garment without removing the garment from the wearer or restricting the wearer's position for diaper changes or access to a toilet. Since the absorbent article is compatible with a plurality of garments, such benefits remain regardless of climate, season, or stylistic choices.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### TECHNICAL FIELD

This application relates to absorbent garments, specifically diapers, worn by infants, children, and incontinent individuals.

### BACKGROUND

The following is a tabulation of some prior art that presently appears relevant:

### U.S. Patents

| Patent | Kind | | |
|---|---|---|---|
| Number | Code | Issue Date | Patentee |
| 6334858 | B1 | Jan. 1, 2002 | Rönnberg et al |
| 7794442 | B2 | Sep. 14, 2010 | Roehrl et al. |
| 8449518 | B2 | Feb.11, 2010 | Allison-Rogers |
| 6478787 | B1 | Nov. 12, 2002 | Ihrfelt et al. |
| 4964860 | B1 | Jan. 18, 1994 | Gipson et al. |
| 5971970 | A | Oct. 26, 1999 | Carlbark et al. |
| 5549593 | A | Aug. 27, 1996 | Ygge et al. |
| 6432099 | B2 | Aug. 13, 2002 | Rönnberg |
| 9554952 | B2 | Jan. 31, 2017 | Rönnberg et al. |
| 5135522 | A | Aug. 4, 1992 | Fahrenkrug et al. |
| 10258518 | B2 | Apr. 16, 2019 | Carlén et al. |
| 4964860 | A | Oct. 23, 1990 | Gipson et al. |
| 9585799 | B2 | Mar. 7, 2017 | Nordlinder et al. |
| 9867412 | B2 | Jan. 16, 2018 | Hansson et al. |
| 8821467 | B1 | Sep. 2, 2014 | Minella |
| 6764479 | B2 | Jul. 20, 2004 | Kusibojoska et al |
| 6645188 | B2 | Nov. 11, 2003 | Kusibojoska et al |
| 6981968 | B2 | Jan. 3, 2006 | Kusibojoska et al |
| 4909802 | A | Mar. 20, 1990 | Ahr et al. |
| 6955668 | B2 | Oct. 18, 2005 | Almberg et al. |
| 6342050 | B1 | Jan. 29, 2002 | Rönnberg et al. |
| 7850673 | B1 | Dec. 14, 2010 | Carlbark et al. |
| 9211220 | B2 | Dec. 15, 2015 | Schambon |
| 5368585 | A | Nov. 29, 1994 | Dokken |
| H1440 | H | May 2, 1995 | New et al. |
| 8992496 | B2 | Mar. 31, 2015 | Bäck |
| 6626882 | B2 | Sep. 30, 2003 | Hjorth |
| 7195622 | B2 | Mar. 27, 2007 | Lindström |
| 7347848 | B2 | Mar. 25, 2008 | Fernfors |
| 4728326 | A | Mar. 1, 1988 | Gilles |
| 8721611 | B2 | May 13, 2014 | Elfsberg |
| 6494873 | B2 | Dec. 17, 2002 | Karlsson et al. |
| 9095480 | B2 | Aug. 4, 2015 | Breeden et al. |
| 3618608 | A | Nov. 9, 1971 | Brink |
| 6648871 | B2 | Nov. 18, 2003 | Kusibojoska et al |
| 7008410 | B2 | Mar. 7, 2006 | Gustin et al. |
| 8181278 | B2 | May 22, 2012 | Odorzynski et al. |
| 3653381 | A | Apr. 4, 1972 | Warnken |
| 5704933 | A | Jan. 6, 1998 | Fell et al. |
| 5669901 | A | Sep. 23, 1997 | LaFortune et al. |
| 3890973 | A | Jun. 24, 1975 | Davis et al. |
| D751697 | S1 | Mar. 15, 2016 | Savea |

### U.S. Patent Application Publications

| Publication | Kind | Publication | |
|---|---|---|---|
| Number | Code | Date | Applicant |
| 20110184372 | A1 | Jul. 28, 2011 | Östlin et al. |
| 20140068839 | A1 | Mar. 13, 2014 | Steele et al. |
| 20070299418 | A1 | Dec. 27, 2007 | Vartiainen |
| 20100036336 | A1 | Feb. 11, 2010 | Bogdanova et al |
| 20020091369 | A1 | Jul. 11, 2002 | Hansson |
| 20070066951 | A1 | Mar. 22, 2007 | LaVon et al. |
| 20080097369 | A1 | Apr. 24, 2008 | Melander |
| 20060247598 | A1 | Nov. 2, 2006 | Roehrl et al. |
| 20070049897 | A1 | Mar. 1, 2007 | LaVon et al. |
| 20070066953 | A1 | Mar. 22, 2007 | LaVon et al. |
| 20030083634 | A1 | May 1, 2003 | Fernfors et al. |
| 20040082932 | A1 | Apr. 29, 2004 | Lauritzen |
| 20060167432 | A1 | Jul. 27, 2006 | Sigari |
| 20050192555 | A1 | Sep. 1, 2005 | Thomas |
| 20030120253 | A1 | Jun. 26, 2003 | Wentzel |

### Foreign Patent Documents

| Foreign Doc. | Country | Kind | | Application or |
|---|---|---|---|---|
| Number | Code | Code | Publication Date | Patentee |
| 0364454 | EP | B1 | Apr. 25, 1990 | Ygge et al. |
| 2019880 | CA | C | Jan. 18, 1991 | Gipson et al. |
| 0506745 | EP | B1 | Oct. 7, 1992 | Bennett et al. |
| 512761 | SE | C2 | Feb. 28, 1997 | Rönnberg et al. |
| 514370 | SE | C2 | Oct. 24, 1997 | Carlbark et al. |
| 1988007335 | WO | A1 | Oct. 6, 1988 | Ygge et al. |
| 0409307 | EP | A2 | Jan. 23, 1991 | Gipson et al. |
| 1991008725 | WO | A1 | Dec. 7, 1990 | New et al. |
| 2003015685 | WO | A1 | Aug. 20, 2001 | Hermansson et al. |
| 508028 | SE | C2 | May 12, 1993 | Carlbark et al. |
| 2009043101 | WO | A1 | Apr. 9, 2009 | Allison-Rogers |
| 2328478 | ES | T3 | Nov. 13, 2009 | Rohrl et al. |
| 1024774 | EP | B2 | Jun. 27, 2007 | Carlbark et al. |
| 267940 | FR | A1 | Feb. 5, 1993 | Najib |
| 2011167352 | JP | A | Sep. 1, 2011 | Kato et al. |
| 3548176 | JP | B2 | Jul. 28, 2004 | Karubaruku et al. |
| 2015069206 | WO | A1 | May 14, 2015 | Ipek |
| 2004069122 | WO | A1 | Sep. 19, 2004 | Nawata et al. |
| 514909 | SE | C2 | May 14, 2001 | Forgar |
| 2010020990 | WO | A2 | Feb. 25, 2010 | Yacobson et al. |
| 2002325796 | JP | A | Nov. 12, 2002 | Fujimoto et al. |
| 102846435 | CN | A | Jan. 2, 2013 | P lun bei li et al. |
| 102846435 | CN | B | Dec. 14, 2016 | P lun bei li et al. |
| 10338607 | DE | A1 | Mar. 17, 2005 | Röhrl et al. |
| 100221391 | KR | B1 | Sep. 15, 1999 | New et al. |
| 2273201 | AU | A | Jun. 25, 2001 | Lauritzen |
| 1295092 | CA | C | Feb. 4, 1992 | Ahr et al. |
| 2014057974 | WO | A1 | Apr. 17, 2014 | Sasayama et al. |
| 2002022065 | WO | A1 | Mar. 21, 2002 | Gustin et al. |
| 2006135281 | WO | A1 | Dec. 21, 2006 | Karlsson |
| 2029079 | EP | B1 | Aug. 8, 2012 | Hermansson |
| 2170307 | CA | A1 | Aug. 25, 1996 | Schmidt |
| 0638304 | EP | A1 | Feb. 15, 1995 | Schmitz |
| 2008037283 | WO | A1 | Apr. 3, 2008 | Winqvist et al. |
| 0696911 | EP | B2 | Oct. 10, 2001 | Carlbark et al. |
| 103052372 | CN | A | Apr. 17, 2013 | Hiroto |
| 10337537 | DE | A1 | Feb. 24, 2005 | Röhrl et al. |
| 2416385 | RU | C1 | Apr. 20, 2011 | Bekk et al. |
| 4975811 | JP | B2 | Jul. 11, 2012 | Herumanson |
| 1022684 | BE | B1 | Jul. 15, 2016 | Annick De |
| 2002024134 | WO | A1 | Mar. 28, 2002 | Rönnberg et al |
| 3611684 | JP | B2 | Jan. 19, 2005 | Kato et al. |
| 3376249 | JP | B2 | Feb. 10, 2003 | Nakaoka et al. |
| 516020 | SE | C2 | Nov. 12, 2001 | Karlsson et al. |
| 1142547 | EP | A1 | Oct. 10, 2001 | Damaghi et al. |
| 203885723 | CN | U | Oct. 22, 2014 | Xuechenfang |
| 203040706 | CN | U | Jul. 10, 2013 | Xuxiaohua |

### Nonpatent Literature Documents

“Drop flap nappy system PDF sewing pattern, nappy diaper belt and wrap, doth diaper nappy, elimination communication, potty training” sold by LittleBunnyBear on Etsy.com
“Drop-Flap Diaper” and “Holding Trousers” sold on windelmanufaktur.com
“Mokomidi Diaper-Free Fleece Pants” sold on mokoshop.eu
“Flaparaps” sold on bornready.eu
“EcaPants” sold on ecwear.com
“rECtangle diaper covers” sold on ecwear.com
“Li'l Baby Chaps” sold on ecwear.com
“Hold-off sets” and “Cowboy pants” sold on Emilino.de
“Windfall 6Pcs/Pack Adjustable Baby Diaper Fastener Nappy Fixed Belt Infant Cloth Holder” sold on Walmart.com
“Adjustable Nappy Diaper belt, prefold belt, cloth nappy belt, elimination communication clothing, nappy free baby clothing, EC gear, baby” sold by LittleBunnyBear on Etsy.com
“Baby Diaper Fastener 4 Hook Safety Nappy Fixing Belt” sold on us.patpat.com
“Baby Nappy Cloth Diaper Buckle Fasteners Changing Nappies Fixed Belt Supplies” sold on Walmart.com
“Organic Cotton Diaper Belt” sold by NaturallyDiaperFree on Etsy.com
“Wool Diaper Belt” sold on babeegreens.com
“Diaper Fixed Belts” sold on Joom.com
“Wool Diaper Belt” sold on asmallgreenfootprint.com
“2 Pack Overnight/Trainer great for EC” sold by diapercircus on Etsy.com

It is common practice in Western society to place infants and young children in absorbent garments, typically diapers, at all times for the containment of bodily excretions. However, the practice of infant potty training, also known as elimination communication (EC), is widely practiced around the globe, particularly in areas with restricted access to disposable diapers. This practice involves learning and responding to an infant's cues that indicate the infant's need to eliminate and offering the infant an appropriate receptacle for urine or feces. The practice can begin as young as birth and leads to a toilet-independent child at an age typically younger than infants who are diapered full-time and then potty trained at a later age. The practice of EC has gained popularity in Western society in addition to being practiced in other parts of the world. Many caregivers who practice EC use conventional diapers in addition to offering the toilet to their child.

The most common type of diaper currently used for infants and children is a disposable diaper having a longitudinal direction and a lateral direction and comprising of a front part, a rear part, an intermediate crotch part, and an absorbent unit for liquids. Typically, the diaper has a waist belt section on the rear-most edge of the rear part and on the front-most edge of the front part, with waist straps extending laterally from the waist of the rear portion. The diaper-wearer is placed in the dorsal recumbent position for diapering, the rear portion is placed below the wearer's buttocks, and the rear waist belt is aligned with the wearer's natural waist. The front portion is then drawn upward between the wearer's legs so that the front waist belt also aligns with the wearer's waist, and then the rear waist straps are brought around the wearer's waist and connected to the front part via fastening means, typically hook and loop.

Disposable diapers and cloth diapers of this kind have the following drawbacks:
1. After achieving mobility, many children are no longer content to lie on their backs for diaper changes, frequently rolling away from their caregiver and hindering the diaper-changing process. Additionally, many adult incontinent individuals have difficulty being diapered in the dorsal recumbent position.
2. Conventional diapers are inconvenient for caregivers practicing EC or potty training, as they pose a barrier to the toilet that must be removed and replaced, possibly along with other layers of clothing, during the process of offering the toilet to the child.

Belt diapers are a form of diaper that aim to solve these issues. Such diapers comprise a hip belt that forms a hip opening of the diaper which is closed in the peripheral direction, and which can be typically opened and closed on top of itself on at least one location using first and second fastening devices. Such a diaper further comprises a front part, a rear part, an intermediate crotch part, and an absorbent unit for liquids, wherein the diaper main part can be detachably fixed, at the longitudinal end of its front part or rear part, to the hip belt via third fastening devices, such that the main diaper part can be pulled between the wearer's legs after application of the hip belt, and detachably fix the free longitudinal end of the diaper main part to the hip belt.

Embodiments of belt diapers previously described have at least one or more of the following disadvantages:
1. If the belt is permanently attached to the front of the diaper, the diaper can only be opened from the rear and pulled forward to offer the toilet to the wearer. In this position, the diaper obstructs a male wearer's penis, making it difficult for a caregiver to assist the wearer to aim his urine into the toilet. Examples of this type include U.S. Pat. No. 6,764,479 to Kusibojoska et al. (2004), U.S. application 20080097369 to Melander (2008), “Flaparaps” sold on bornready.eu, among others.
2. If the belt is permanently attached to the rear of the diaper, the diaper can only be opened from the front and pulled backward to offer the toilet to the wearer. If the wearer has a bowel movement with this arrangement, the diaper impedes access to the wearer's rear end for cleaning. Examples of this type include U.S. Pat. No. 8,821,467 to Minella (2014), U.S. application 20100036336 to Bogdanova et al. (2010), U.S. Pat. No. 6,764,479 to Kusibojoska et al. (2004), U.S. Pat. No. 6,955,668 to Almberg et al. (2005), U.S. patent 9211220 to Schambon (2015), “Drop-Flap Diaper” sold on windelmanufaktur.com, “Hold-off sets” sold on Emilino.de, among others.
3. If the belt is permanently attached in any position or must be attached to the diaper before the belt can be placed on the wearer, then both diaper and belt must be removed every time the wearer wets or soils the diaper, causing inconvenience to the caregiver. Examples of this type include U.S. Pat. No. 5,135,522 to Fahrenkrug et al. (1992), U.S. application 20020091369 to Hansson (2002), U.S. Pat. No. 4,909,802 to Ahr et al. (1990), U.S. Pat. No. 6,342,050 to Ronnberg et al. et al. (2002), U.S. application 20070299418 to Vartiainen (2007), among others.
4. The diaper requires hook and loop closures to fasten to the belt, a method of fastening that often results in a shorter product lifespan than other closure methods. Examples of this type include U.S. Pat. No. 5,549,593 to Ygge et al. (1996), U.S. Pat. No. 8,449,518 to Allison-Rogers (2010), U.S. Pat. No. 5,971,970 to Carlbark et al. (1999), U.S. Pat. No. 9,554,952 to Rönnberg et al. (2017), among others.
6. The diaper requires central fasteners along a central longitudinal axis in the front and/or rear of the diaper in addition to lateral fasteners in the front and rear to be successfully attached to the belt, causing inconvenience to the caregiver. Examples of this type include U.S. Pat. No. 6,334,858 to Rönnberg et al. (2002), “Holding Trousers” sold on windelmanufaktur.com, among others.
7. The diaper is disposable; disposable diapers are costly for caregivers and place a strain on the environment. Examples of this type include U.S. Pat. No. 7,794,442 to Roehrl et al. (2010), U.S. Pat. No. 8,449,518 to Allison-Rogers (2010), W.I.P.O. publication 1991008725 to New et al. (1990), U.S. Pat. No. 6,432,099 to Ronnberg (2002), Spanish publication 2328478 to Rohrl et al. (2009), among others.
8. The diaper can only be attached to an article of clothing with sleeves for the legs, which means the diaper cannot be used by wearers in warmer climates or seasons. An example of this type includes Chinese publication 203885723 to Xuechenfang (2014).
9. Neither the belt nor the diaper has means of covering the wearer's legs, leaving the legs bare unless they are covered with outerwear such as pants, leg warmers, a bodysuit, or other clothing, which inconveniences the caregiver during diaper changes and when offering the toilet to the wearer. Examples of this type include U.S. Pat. Nos. 6,334,858 and 6,432,099 to Rönnberg et al. (2002), U.S. Pat. No. 5,549,593 to Ygge et al. (1996), U.S. application 20110184372 to Ostlin et al (2011), U.S. Pat. No. 7,794,442 to Roehrl et al. (2010), U.S. Pat. No. 8,449,518 to Allison-Rogers (2010), W.I.P.O. publication 1991008725 to New et al. (1990), U.S. Pat. No. 5,971,970 to Carlbark et al. (1999), U.S. Pat. No. 9,554,952 to Rönnberg et al. (2017), U.S. application 20060167432 to Sigari (2006), among others.
10. The diaper is designed for use in adult incontinent individuals and does not adequately reflect the needs of caregivers practicing EC or potty training with their infants and children. An example of this type includes U.S. application 20050192555 to Thomas (2005).
11. The belt is intended to wrap around the waist of the diaper after the diaper is placed on the wearer to inhibit the diaper's removal by the wearer or to aid in securing it; such belt arrangements obstruct the practice of EC. An example of this type is U.S. application 20140068839 to Steele et al. (2014).
12. The belt is an elastic ring meant to hold up a separate diaper without a means of fastening the diaper to the belt. Such belt arrangements do not securely hold the diaper and are prone to leaks. Examples of this type include “rECtangle diaper covers” sold on ecwear.com, “Li'l Baby Chaps” sold on ecwear.com, “Windfall 6Pcs/Pack Adjustable Baby Diaper Fastener Nappy Fixed Belt Infant Cloth Holder” sold on Walmart.com, “Adjustable Nappy Diaper belt, prefold belt, cloth nappy belt, elimination communication clothing, nappy free baby clothing, EC gear, baby” sold by LittleBunnyBear on Etsy.com, “Baby Diaper Fastener 4 Hook Safety Nappy Fixing Belt” sold on us.patpat.com, among others.

### SUMMARY OF THE EMBODIMENTS

In accordance with one embodiment, a series of garments comprises of an absorbent article and compatible secondary articles. The absorbent article comprises a front part (21), a rear part (22), an intermediate crotch part (24), an absorbent unit (23), an inner facing material (26), a liquid impervious top sheet (38), and both lateral and rear elastic elements (32A, 32B, 34, 36). Each of the compatible articles comprises of a waist belt with a front part (40, 52, 68, 82, 96) and a rear part (42A, 42B, 54, 70, 85) among other possible features such as sleeves for legs (62A-62B, 86A-86B), a skirt (72, 90), or a shirt portion (80, 84, 91), such that when such a compatible article, hereby referred to as the garment of choice, is placed on a wearer, the wearer's groin and buttocks are uncovered. Fastening devices are positioned on the front parts of both the absorbent article (30A-30B) and the waist belts of the compatible secondary articles (44A, 44B, 56A, 56B, 78A, 78B, 92B) and on the rear parts of both the absorbent article (28A—28D) and the waist belts of the compatible secondary articles (46A—46D, 66A—66D, 76A—76D, 94C—94D). This enables the garment of choice to be placed on the wearer and remain in place while the absorbent article is fully attached to the waist belt to cover the wearer's exposed groin and buttocks, or while the absorbent article is fully or partially detached from the waist belt, or while the absorbent article is replaced with a secondary, identical absorbent article. Thus, the absorbent article can be quickly and easily attached to, detached from, partially attached to, or partially detached from the garment of choice without removing the garment of choice from the wearer or restricting the wearer's position for diaper changes or access to a toilet. Since the absorbent article is compatible with multiple variations of compatible secondary articles, including garments with sleeves for the legs, a skirt, or a shirt portion, these benefits are enjoyed regardless of climate, season, or stylistic choices.

### BRIEF DESCRIPTION OF THE DRAWINGS

FIG. 1 illustrates an interior view of an absorbent article (diaper) when the diaper is laid flat.

FIG. 2 illustrates an exterior view of the absorbent article (diaper) when the diaper is laid flat.

FIG. 3 illustrates a fully detachable waist belt when it is opened and laid flat.

FIG. 4 illustrates a perspective view of the waist belt when it is closed.

FIG. 5 illustrates the diaper partially attached to the waist belt at the front fastening devices for both the waist belt and the diaper so that the rear part of the diaper hangs freely.

FIG. 6 illustrates the diaper partially attached to the waist belt at the rear fastening devices for both the waist belt and the diaper so that the front part of the diaper hangs freely.

FIG. 7 illustrates a rear view of the diaper fully attached to the waist belt, which is positioned on a wearer.

FIG. 8 illustrates a front view of the diaper fully attached to the waist belt, which is positioned on a wearer.

FIG. 9 illustrates a perspective view of the front of a pant garment with waist belt.

FIG. 10 illustrates a perspective view of the rear of the pant garment with waist belt.

FIG. 11 illustrates a front view of the diaper fully attached to the waist belt of the pant garment, which is positioned on a wearer.

FIG. 12 illustrates a rear view of the diaper fully attached to the waist belt of the pant garment, which is positioned on a wearer.

FIG. 13 illustrates a perspective view of the front of a skirt garment with waist belt.

FIG. 14 illustrates the skirt garment with a portion of the skirt material lifted up to reveal the positions of the fastening devices, which are hidden underneath the skirt fabric when the garment is positioned on a wearer.

FIG. 15 illustrates a rear view of the diaper fully attached to the waist belt of the skirt garment, which is positioned on a wearer.

FIG. 16 illustrates a pattern shape that is used to cut out the inner facing material and liquid impervious top sheet of the diaper in one embodiment.

FIG. 17 illustrates a pattern shape that is used for crafting the pant garment in one embodiment.

FIG. 18 illustrates a front view of the diaper fully attached to a shirt garment with waist belt, which is positioned on a wearer.

FIG. 19 illustrates a rear view of the diaper fully attached to a bodysuit garment with waist belt, shirt portion, and sleeves for legs, which is positioned on a wearer.

FIG. 20 illustrates a perspective view of a dress garment with waist belt, skirt, and shirt portion; part of the skirt of the dress garment is lifted to reveal fastening devices.

### DRAWINGS—REFERENCE NUMERALS

121. Front part (diaper)
122. Rear part (diaper)
123. Absorbent unit
124. Intermediate crotch part
126. Inner facing material
128. (A-D) Rear fastening devices (diaper)
130. (A-B) Front fastening devices (diaper)
132. (A-B) Lateral elastic elements
134. Elastic element in inner facing material
136. Elastic element in liquid impervious top sheet
238. Liquid impervious top sheet
340. Front part (belt)
342. (A-B) Rear parts (belt)
344. (A-B) Front fastening devices (belt)
346. (A-D) Rear fastening devices (belt)
348. (A-D) Belt closure devices
350. (A-D) Elastic elements (belt)
952. Front part (pant belt)
954. Rear part (pant belt)
956. (A-B) Front fastening devices (pant garment)
958. (A-B) Upper leg holes (pant garment)
960. (A-B) Lower foot holes (pant garment)
962. (A-B) Sleeves for legs (pant garment)
964. (A-B) Elastic elements (pant garment)
1066. (A-D) Rear fastening devices (pant garment)
1368. Front part (skirt belt)
1370. Rear part (skirt belt)
1372. Skirt material (skirt garment)
1374. (A-B) Elastic elements (skirt garment)
1376. (A-D) Rear fastening devices (skirt garment)
1378. (A-B) Front fastening devices (skirt garment)
1880. Shirt portion (shirt garment)
1881. Neck hole (shirt garment)
1882. Waist belt (shirt garment)
1883. (A-B) Sleeves for arms (shirt garment)
1984. Shirt portion (bodysuit garment)
1985. Waist belt (bodysuit garment)
1986. (A-B) Sleeves for legs (bodysuit garment)
1987. Undershirt
1988. Neck hole (bodysuit garment)
1989. (A-B) Shoulder fastening devices (bodysuit garment)
2090. Skirt material (dress garment)
2091. Shirt portion (dress garment)
2092. (B) Front fastening device (dress garment)
2094. (C-D) Rear fastening devices (dress garment)
2096. Waist belt (dress garment)
2098. (A-B) Sleeves for arms (dress garment)
2099. Neck hole (dress garment)

### DETAILED DESCRIPTION OF SPECIFIC EMBODIMENTS

It is the aim of the present application to solve the disadvantages discussed in the background. Thus, some embodiments provide a caregiver a choice between unfastening a diaper from the front or the rear in the process of offering the toilet to a wearer. Some embodiments enable the diaper to be changed without removing a compatible garment from the wearer in the process. Some embodiments of the diaper will be compatible with a belt that leaves the wearer's legs bare as well as a pant garment that covers the wearer's legs, enabling the diaper to be worn in any climate or season. Some embodiments will employ snap closures instead of hook and loop, a sturdier and longer-lasting closure method, particularly for washable garments. Some embodiments will use four closures in order to successfully attach the diaper to the waist belt of the compatible garment, two fasteners arranged laterally in the front and two arranged laterally in the rear. Some embodiments are washable and reusable, making the embodiment a cheaper choice for caregivers and less of a burden on the environment. Some embodiments will be specifically designed for use on infants with caregivers practicing EC. These and other benefits of one or more aspects will become apparent from a consideration of the ensuing description and accompanying drawings.

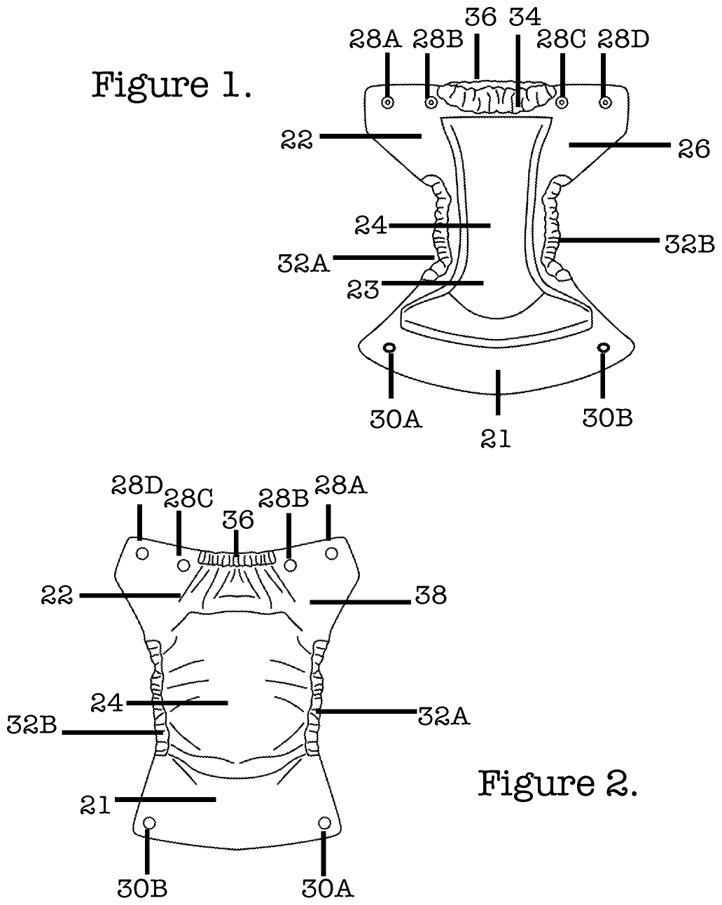

### Description of the Absorbent Article—FIGS. 1, 2, and 16

FIG. 1 shows a view of the interior of an embodiment of an absorbent article, most typically referred to as a diaper or nappy, when the diaper is laid flat. FIG. 2 shows a view of the exterior of the diaper when the diaper is laid flat. Both FIGS. 1 and 2 indicate a front part 21, a rear part 22, and an intermediate crotch part 24. An outer layer of the diaper shown in FIG. 2 is a liquid impervious top sheet 38. In this embodiment, the top sheet 38 is cut from a length of polyurethane laminate fabric (PUL), a breathable, water-repellent fabric often used in the manufacture of cloth diapers. Other materials suitable for this purpose would include wool, plastic, certain kinds of fleece, among others. The fabric for this layer is cut into a fitted diaper shape, a pattern for which is shown in FIG. 16. FIG. 16 indicates that in this embodiment, the front part 21 of the diaper will be cut narrower than the rear part 22, with the intermediate crotch part 24 tapering between the two widths. An inner facing material 26 shown in FIG. 1 is cut in the same pattern shape as shown in FIG. 16. In this embodiment, the inner facing material 26 is cut from a length of fleece and sewn to the liquid impervious top sheet 38. Other suitable materials for the inner facing material would be suede, microfleece, terrycloth, birdseye cotton, diaper twill, among others.

As shown in FIG. 1, an absorbent unit 23 is sewn to the inner facing material 26. In this embodiment, the absorbent unit is made of folded layers of 100% cotton birdseye fabric. This embodiment uses eight to ten layers of birdseye cotton; however, different materials would be suitable for the absorbent unit in different numbers of layers (for example, diaper twill, terrycloth, absorbent microfiber, among others).

The diaper shown in FIGS. 1 and 2 is given further shaping in this embodiment by adding lateral elastic elements 32A and 32B as well as rear elastic elements in the inner facing material 34 and the liquid impervious top sheet 36. Elastic ribbon is used for this embodiment, but other forms of elastic, such as fold-over elastic, are suitable. As seen in FIG. 1, sewing separate elastic elements into the rear part of the inner facing material 34 and the rear part of the liquid impervious top sheet 36 provides the diaper of this embodiment with a pocket between the inner facing 26 and the top sheet 38 into which additional absorbent units could be inserted to increase the diaper's absorbency. In one embodiment, this is achieved by sewing the absorbent unit 23 to a liquid impervious inner facing material 26 and then removing the inner facing material directly beneath the absorbent unit so that liquid may pass through the absorbent unit 23 and into the pocket between the inner facing 26 and top sheet 38. In other embodiments, the inner facing material 26 and the absorbent unit 23 are both made from absorbent material, rendering the process of cutting the inner facing material unnecessary. In other embodiments, the absorbent unit 23 is fully detached from the inner facing material 26 and inserted directly into the pocket formed by the inner facing 26 and top sheet 38. Other methods exist for crafting such a pocket.

The embodiment of FIGS. 1 and 2 also has four rear fastening devices 28A-28D and two front fastening devices 30A and 30B. In this embodiment, the fastening devices are snaps with male snaps located at the lateral edges of the rear part 22 of the diaper and female snaps located at the lateral edges of the front part 21 of the diaper. Although snaps are used in this embodiment, other closure devices, such as buttons or hook and eye, may be used instead.

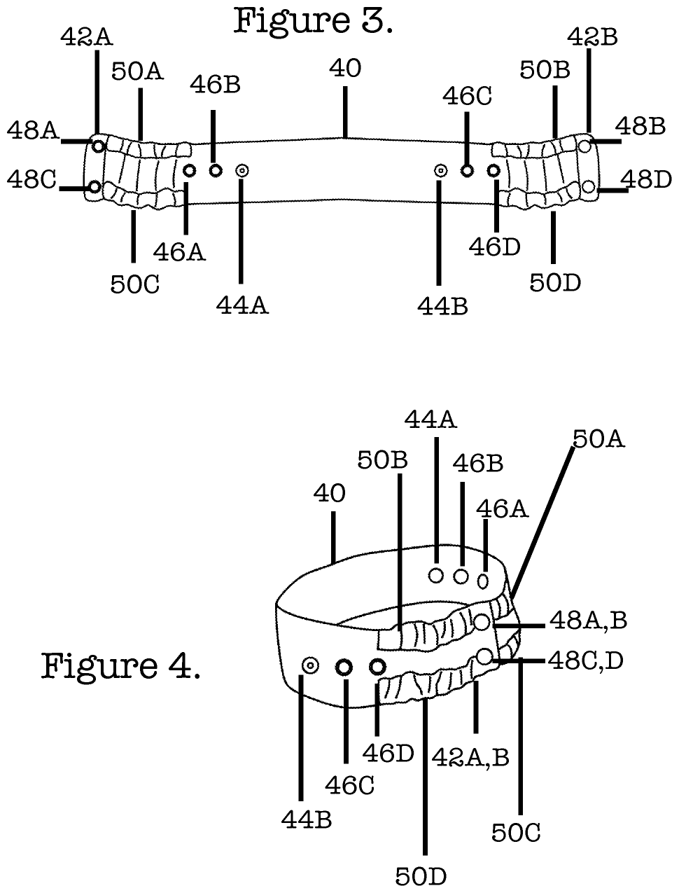

### Description of the Waist Belt—FIGS. 3 and 4

FIG. 3 shows a detached waist belt that is open and laid flat. FIG. 4 shows the same waist belt that has been closed using belt closure devices 48A-48D. As can be seen in FIG. 3, rear parts 42A and 42B of the belt are divided in half when the belt closure devices 48A-48D are released. A front part 40 of the belt shown in FIG. 3 is then in the center of the belt when laid flat. In FIG. 4, the two halves of the rear part 42A and 42B have been joined by the belt closure devices 48A-48D, which have been fastened. This places the rear part 42 directly behind the front part 40.

In this embodiment, the waist belt is comprised of fleece, but other materials such as wool or PUL could be used. The belt is shaped by cutting a rectangle of fleece of dimensions 10.16 centimeters by 53.34 centimeters, although other sized rectangles would be appropriate for embodiments intended for larger or smaller individuals. This rectangle is folded on itself along its length to form a rectangle measuring 5.08 centimeters by 53.34 centimeters, which is then sewn along the raw edges. Elastic elements 50A-50D, shown in both FIG. 3 and FIG. 4, are then added to provide shaping.

The belt of FIGS. 3 and 4 also has front fastening devices 44A and 44B and rear fastening devices 46A-46D. These devices are snaps in this embodiment, with the front snaps 44A and 44B being male snaps and the rear snaps 46A-46D being female snaps. The belt closure devices 48A-48D shown in FIGS. 3 and 4 are also made of snaps, with two male snaps 48B and 48D on one half of the rear part 42B and two female snaps 48A and 48C on the other half of the rear part 42A so that the male and female snaps may be brought together to connect the two halves as shown in FIG. 4. Other closure devices besides snaps could also be used in other embodiments.

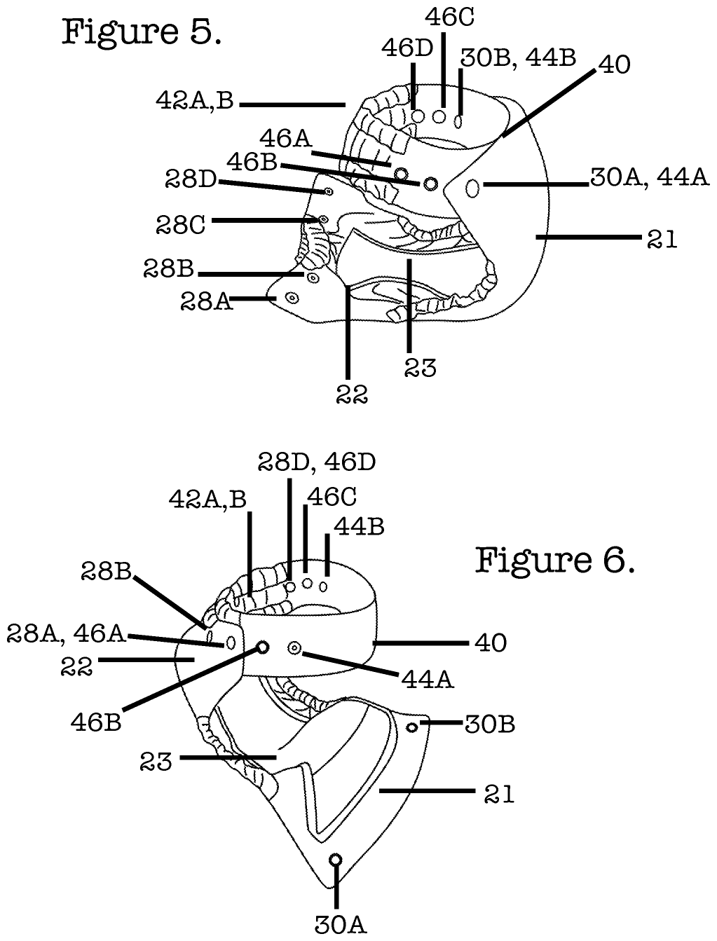

### Compatibility of Absorbent Article and Waist Belt—FIGS. 5-8

FIG. 5 shows how the waist belt can be fastened to the diaper by connecting the front fastening devices on the belt 44A and 44B to the front fastening devices on the diaper 30A and 30B, respectively. The belt is shown as being closed in FIG. 5 and the rear fastening devices of the diaper 28A-28D are not connected to any of the rear fastening devices of the belt 46A-46D. In this embodiment, the front fastening devices of the belt 44A and 44B are male snaps, which are connected to female snaps that are the front fastening devices of the diaper 30A and 30B. In this position, the front part of the diaper 21 is snugly resting against the front part of the belt 40 while the rear part of the diaper 22 hangs freely from the rear part of the belt 42, allowing the absorbent unit 23 to be visible.

FIG. 6 shows how the waist belt can be fastened to the diaper by connecting two of the rear fastening devices on the belt, in this case 46A and 46D, to two of the rear fastening devices on the diaper, in this case 28A and 28D, respectively. The belt is shown as being closed in FIG. 6 and the front fastening devices of the diaper 30A and 30B are not connected to the front fastening devices of the belt 44A and 44B. In this embodiment, the rear fastening devices of the belt 46A-46D are female snaps, only two of which need be connected to two of the male snaps that are the rear fastening devices of the diaper 28A-28D. Two of the rear snaps of the diaper, in this case 28B and 28C, are not snapped to any other snap; additionally, two of the rear snaps of the belt, in this case 46B and 46C, are not snapped to any other snap. This is due to the fact that for this embodiment, only two snaps are required to satisfactorily connect the rear of the diaper 22 to the rear of the belt 42. In this position, the rear part of the diaper 22 is snugly resting against the rear part of the belt 42 while the front part of the diaper 21 hangs freely from the front part of the belt 40, allowing the absorbent unit 23 to be visible.

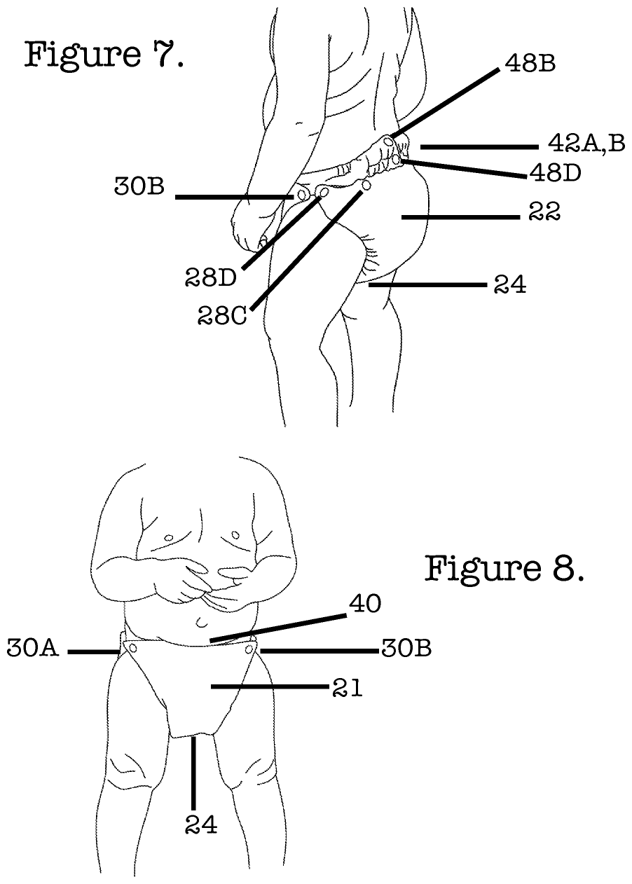

FIG. 7 illustrates a rear view of the diaper fully attached to the waist belt, which is positioned on a wearer. The rear part of the diaper 22 hugs snugly to the rear part of the belt 42. The intermediate crotch part of the diaper 24 is drawn between the wearer's legs and rests snugly against the wearer's body. The belt is closed, being fastened by the belt closure devices 48A-48D. When this embodiment is adjusted properly, the front and rear fastening devices of the diaper, 30B, 28C, and 28D being shown in FIG. 7, rest on the wearer's hips.

FIG. 8 illustrates a front view of the diaper fully attached to the waist belt, which is positioned on a wearer. The front part of the diaper 21 hugs snugly to the front part of the belt 40. The intermediate crotch part of the diaper 24 is drawn between the wearer's legs and rests snugly against the wearer's body. When this embodiment is adjusted properly, the front fastening devices of the diaper 30A and 30B rest on the wearer's hips.

### Operation of Waist Belt and Absorbent Article—FIGS. 1-6

In operation, the diaper and waist belt garment work together to overcome some of the disadvantages of the prior art discussed above. The assembly is put onto the wearer in this embodiment by first securing the separate waist belt of FIGS. 3 and 4 onto the wearer's natural waist above the hips. The front part 40 of the waist belt is centered below the wearer's navel or bellybutton. The two halves of the rear part 42A and 42B are then brought around the wearer's back and secured by fastening the belt closure devices 48A-48D. The rear part 42 should be centered at the wearer's spine above the buttocks.

The diaper shown in FIGS. 1 and 2 can then be attached in one embodiment to the waist belt by first fastening the front fastening devices of the diaper 30A and 30B to the front fastening devices of the belt 44A and 44B, shown in FIGS. 3 and 4, respectively. Then the diaper is drawn between the wearer's legs and up above the wearer's buttocks. At this stage in the operation of this embodiment, the caregiver has several choices for fastening the rear part of the diaper 22 to the rear part of the belt 42. Fastening rear diaper fasteners 28A and 28D to rear belt fasteners 46A and 46D, respectively, will result in the loosest possible fit. Fastening rear diaper fasteners 28A and 28D to rear belt fasteners 46B and 46C, respectively, will result in a tighter fit. Fastening rear diaper fasteners 28B and 28C to rear belt fasteners 46A and 46D, respectively, will result in an even tighter fit. Fastening rear diaper fasteners 28B and 28C to rear belt fasteners 46B and 46C, respectively, will result in the tightest fit. The ability to adjust this embodiment to four different tightness levels offers a great advantage to caregivers of infants and children, as children tend to grow quickly during the diapering years. Although this embodiment provides four tightness levels, other embodiments could have more or less, depending on the age and size of the intended wearer. This would be achieved by adding or removing snaps from either the waist belt or the diaper or both.

Once the two chosen rear fastening devices of the diaper are fastened to the two chosen rear fastening devices of the belt, the diaper is fully secured to the wearer using four fastening devices total. This is advantageous over many prior art references mentioned in the background, which require five, six, or more total fastening devices for the diaper to be fully secured to the belt, an inconvenience to the caregiver.

Alternatively, in another embodiment the diaper can be secured to the belt after applying the belt to the wearer by fastening the chosen rear fastening devices first, and then drawing the diaper between the wearer's legs to fasten the front fastening devices. Alternatively, before the belt is placed around the wearer's body and while it is still open (belt closure devices 48A-48D being unfastened), the front fastening devices of the diaper 30A and 30B can be fastened to the front fastening devices of the belt 44A and 44B. Then the waist belt can be wrapped around the wearer's body and the belt closure devices 48A-48D can be fastened in line with the wearer's spine. Then the diaper can be brought between the wearer's legs in order to fasten the rear fastening devices.

Alternatively, before the belt is placed around the wearer's body, the belt can be closed using the belt closure devices 48A-48D. Then the front fastening devices of the diaper 30A and 30B can be fastened to the front fastening devices of the belt 44A and 44B and two of the rear fastening devices of the diaper can be fastened to two of the rear fastening devices of the belt, creating a pant-like shape. Then the combined garment can be pulled onto the wearer in the manner of a pull-up diaper pant. Alternatively, the belt can initially be placed around the wearer's waist backward, with the rear parts 42A and 42B being joined directly beneath the wearer's navel. Then the chosen rear fastening devices can be employed to attach the rear part of the diaper 22 to the rear part of the belt 42. Next, the wearer or caregiver can rotate the belt until the front of the belt 40 is centered below the wearer's navel before pulling the front part of the diaper 21 between the wearer's legs to fasten the front fastening devices. The previous two methods both allow the wearer to be able to put on the diaper assembly by him or herself.

Other embodiments may have additional methods of attaching the garments to the wearer. The large number of ways to apply the garments to the wearer offers flexibility to the caregiver that most of the prior art lacks. When babies achieve mobility, many are not content to lie still in the dorsal recumbent position for the amount of time required to put on a traditional diaper. Being able to put the diaper on the child in any number of positions, including hands and knees, standing, while being held by the caregiver, and others, is an advantage of this embodiment. The ability for an older child or adult to put the diaper on by him or herself, granting the wearer independence from a caregiver, is also an advantage of this embodiment.

Once the diaper is on the wearer, the diaper can be removed and replaced with an identical second diaper by unfastening the front and rear fastening devices in any order to release the wet or soiled diaper. The waist belt remains in place during this process; the replacement diaper is attached to the waist belt in any one of the manners described above. Provided that the belt is not soiled with feces, the same belt can remain on the wearer for many diaper changes before needing to be washed. The wet or soiled diaper of this embodiment can also be washed and reused.

Since the belt remains on the wearer during diaper changes, removing the diaper of this embodiment is much quicker than with traditional diapers and much of the prior art. A child can remain standing, crawling, walking, cruising, or any number of other positions during the process. Putting the replacement diaper onto the child is also simpler than putting on the first diaper, as the belt is already in place; thus, the second diaper can be put onto the child while standing, crawling, cruising, among other positions. The caregiver can also both detach and replace the diaper with one hand while holding the child with the other. This is advantageous in situations such as a public restroom that does not have a changing pad available for the caregiver's use. This is also advantageous to caregivers who may be physically disabled.

The operation of this embodiment continues with offering the toilet to the wearer. Provided that the diaper is not wet or soiled, the diaper can be partially opened to allow the wearer access to the toilet. FIG. 5 illustrates how the diaper can be unfastened in the rear and pulled forward between the wearer's legs while remaining attached at the front. The wearer can then be placed on a toilet or other receptacle. With the diaper in this position, the caregiver can easily access the wearer's rear end for cleaning after a bowel movement. When the wearer is finished eliminating, the diaper can be brought back between the wearer's legs and refastened in the rear.

FIG. 6 illustrates how the embodiment can be unfastened in the front and pulled backward between the wearer's legs while remaining attached at the rear. The wearer can then be placed on a toilet or other receptacle. This position for the diaper is advantageous for male wearers, as the caregiver can easily access the wearer's penis to assist aiming into the toilet or other receptacle. The male wearer can also remain standing when the diaper is unfastened in the front, allowing him to stand while urinating. When the wearer is finished eliminating, the diaper can be brought back between the wearer's legs and refastened in the front. The ability to quickly and easily open the diaper from the front or the rear provides the caregiver with flexibility and is an advantage to this embodiment.

In the event that a caregiver is not satisfied with the absorbency of the diaper as it is shown in this embodiment, the caregiver has the option of adding absorbency via the pocket described above. Referring back to FIG. 1, the embodiment has a rear elastic element 34 in the inner facing material 26 that is separate from the rear elastic element 36 in the liquid impervious top sheet 38. This creates a pocket into which additional absorbent units may be inserted, allowing the caregiver the flexibility to customize the absorbency of the garment for the needs of the specific wearer and circumstance. For instance, more absorbency may be desired as the child ages, during extended periods of wear such as naps and car rides, or due to the caregiver's preference.

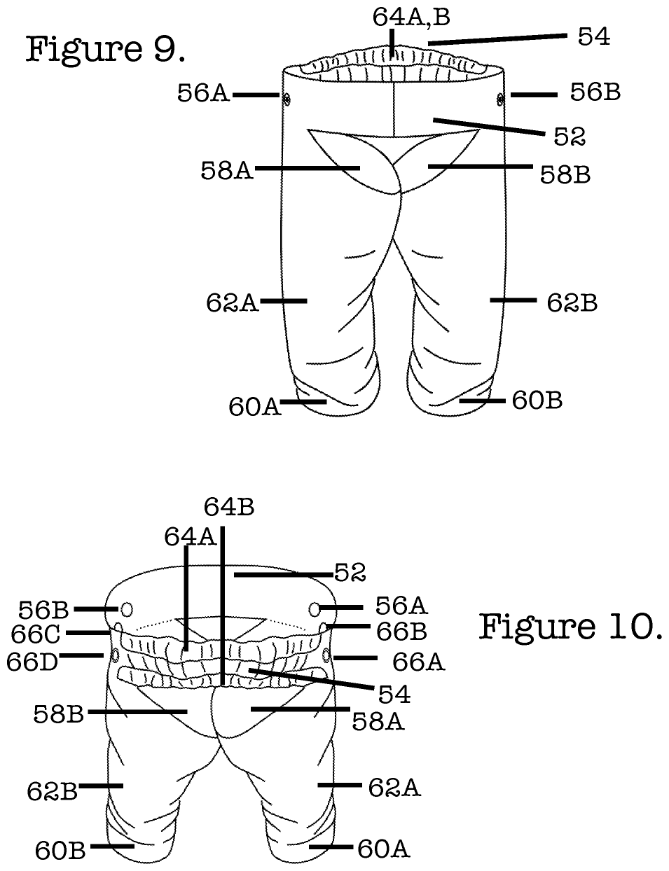

### Alternative Embodiments—FIGS. 9-15 and 17-20

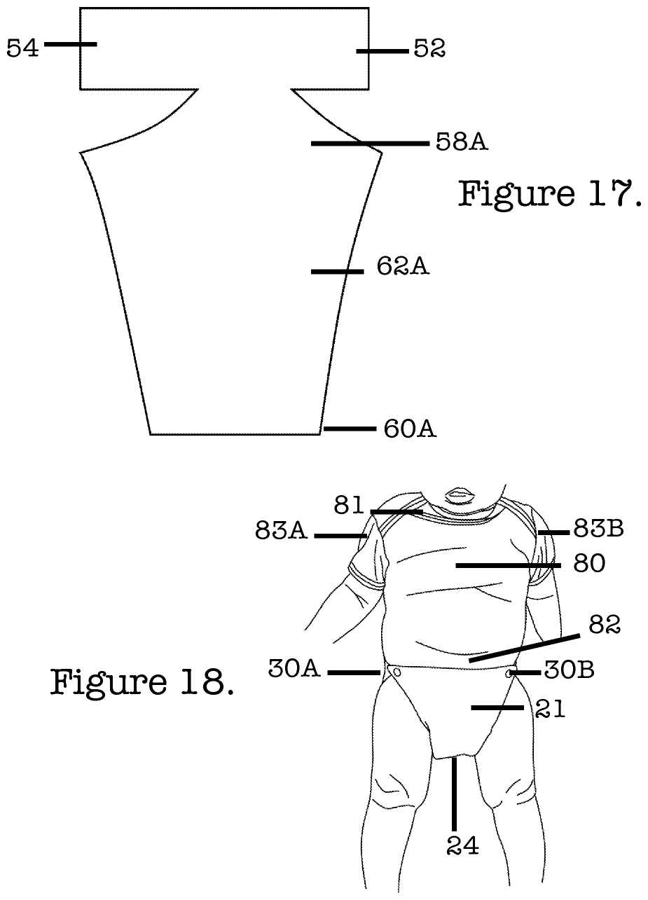

FIG. 9 shows a front view of a detached pant garment with a waist belt and FIG. 10 shows a rear view of the same detached pant garment. In this embodiment, the pant garment is comprised of fleece, but other materials for clothing, such as wool or suede, would be suitable. In this embodiment, the pant garment is shaped by folding a length of fleece in half and cutting two pattern shapes such as the one shown in FIG. 17. These two pattern shapes are sewn right sides together along front parts 52 and along rear parts 54 to form a continuous ring for the waist belt. The waist belt is then folded along its length and sewn in place in the same manner as folding and sewing the waist belt of FIG. 3. Finally, pant legs with upper leg holes 58A-58B, sleeves for legs 62A-62B, and lower foot holes 60A-60B, shown in FIGS. 9 and 10, can be shaped by sewing the lateral edges of each of the individual pattern shapes shown in FIG. 17 starting at the upper leg hole 58A and ending at the lower foot hole 60A. FIGS. 9 and 10 also show the elastic elements 64A and 64B, which are subsequently added to provide shaping.

Fastening devices can also be seen in FIGS. 9 and 10, with front fastening devices of the pant garment being male snaps 56A and 56B in this embodiment. In FIG. 10, rear fastening devices of the pant garment 66A-66D can also be seen, with the rear fastening devices being female snaps in this embodiment.

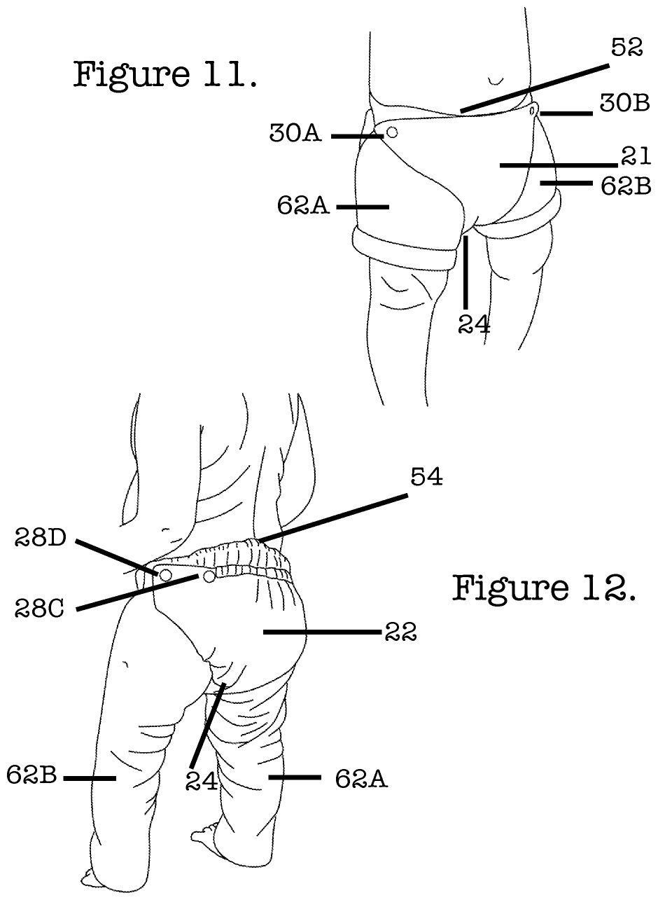

FIG. 11 illustrates a front view of the diaper fully attached to the pant garment, which is positioned on a wearer. The front part of the diaper 21 hugs snugly to the front part of the pant garment 52. The intermediate crotch part of the diaper 24 is drawn between the wearer's legs and rests snugly against the wearer's body. When this embodiment is adjusted properly, the front fastening devices of the diaper 30A and 30B rest on the wearer's hips. In this embodiment, the sleeves for the legs 62A and 62B are cut short enough to be worn as shorts ending just above the wearer's knees, although other lengths and styles could also be crafted.

FIG. 12 illustrates a rear view of the diaper fully attached to the pant garment, which is positioned on a wearer. The rear part of the diaper 22 hugs snugly to the rear part of the pant garment 54. The intermediate crotch part of the diaper 24 is drawn between the wearer's legs and rests snugly against the wearer's body. When this embodiment is adjusted properly, the rear fastening devices of the diaper, 28C and 28D being shown in FIG. 12, rest on the wearer's hips. In contrast to FIG. 11, this embodiment illustrates sleeves for the legs 62A and 62B that are cut long enough to cover the wearer's legs all the way to the heels of the wearer's feet.

FIG. 13 illustrates a perspective view of a skirt garment with waist belt with a front part 68 and a rear part 70. In this embodiment, the waist belt of the skirt garment is comprised of fleece, but other materials such as wool or PUL could be used. The waist belt of the skirt is shaped by cutting a rectangle of fleece of dimensions 10.16 centimeters by 53.34 centimeters, although other sized rectangles would be appropriate for embodiments intended for larger or smaller individuals. The two lateral ends of this rectangle are sewn together to form a ring, with the seam in the rear part 70. A length of skirt material 72 is then cut and gathered around the waist belt of the skirt, which is then partially folded over the raw edge of the skirt material and sewn into place. The skirt fabric in this embodiment is made from a woven knit fabric, although other materials such as cotton or polyester blends would also be sufficient. Elastic elements 74A and 74B are then added to provide shaping in the rear part 70. Rear fastening devices 76A-76D and front fastening devices 78A and 78B are then added to the waist belt. In this embodiment, the fastening devices are applied to the waist belt in such a manner that the skirt material 72 hangs over them.

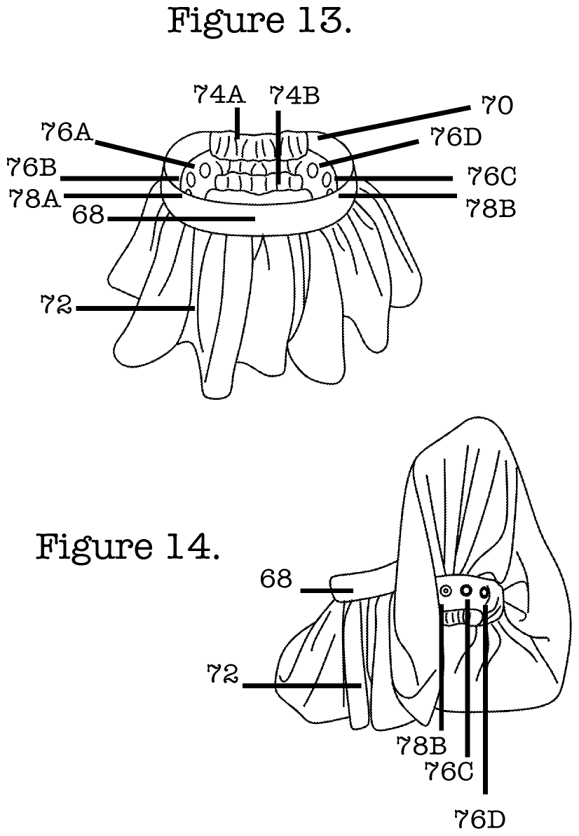

FIG. 14 provides an alternate view of the location of the fastening devices on the skirt garment, which in this embodiment are snaps located on each side of the skirt waist belt between the front part 68 and the rear part 70. FIG. 14 shows one side of the waist belt when the skirt material 72 is lifted up to reveal the concealed rear fastening devices 76C and 76D and front fastening device 78B. In this embodiment, the front fastening device 78B is a male snap and the rear fastening devices 76C and 76D are female snaps. Other fastening devices could also work.

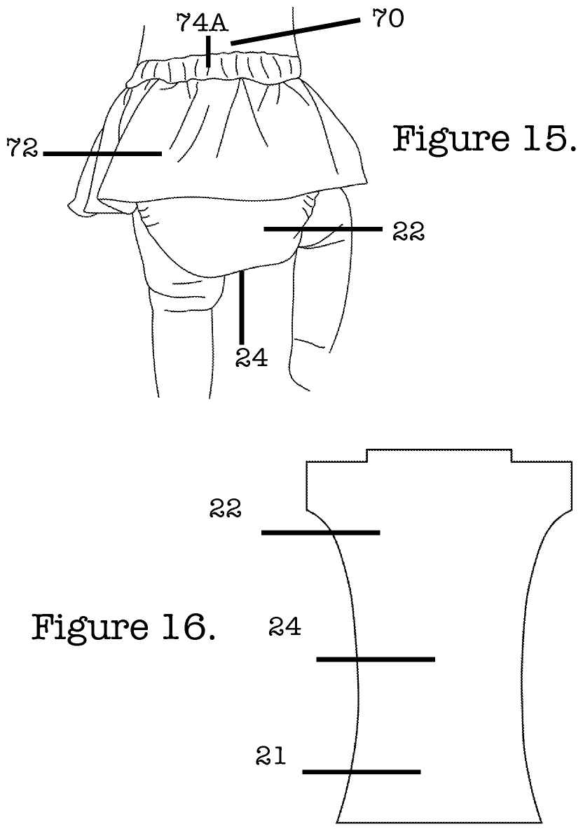

FIG. 15 illustrates a rear view of the diaper fully attached to the skirt garment, which is positioned on a wearer. The rear part of the diaper 22 hugs snugly to the rear part of the waist belt of the skirt garment 70, but since the fastening devices are underneath the skirt material 72, the top portion of the rear part of the diaper 22 is hidden underneath the skirt material 72 along with all of the fastening devices. The intermediate crotch part of the diaper 24 is drawn between the wearer's legs and rests snugly against the wearer's body. Elastic element 74A is visible above the skirt material 72 in the rear part 70, but elastic element 74B is hidden beneath the skirt material 72.

FIG. 18 illustrates a front view of the diaper fully attached to a shirt garment with a waist belt 82, which is positioned on a wearer. The shirt garment is constructed in this embodiment by first fabricating a shirt 80 out of suitable material, for example cotton knit, polyester blend, fleece, or wool. The waist belt 82 is then attached to the lower raw edge of the shirt 80. This is accomplished in this embodiment by cutting a rectangle of fleece of dimensions 10.16 centimeters by 53.34 centimeters, although other sized rectangles would be appropriate for embodiments intended for larger or smaller individuals. The two lateral ends of this rectangle are sewn together to form a ring, which is then folded in half lengthwise with the lower raw edge of the shirt material sandwiched between the raw edges of the waist belt. Similar to the previously described garments, fastening devices and elastic elements are then added to the waist belt 82 of the shirt garment, although these are not shown in FIG. 18.

FIG. 18 illustrates how the shirt garment can be positioned on a wearer, with the diaper fully attached to the waist belt 82 of the shirt garment. The front part of the diaper 21 hugs snugly to the front part of the waist belt 82 of the shirt garment. The intermediate crotch part of the diaper 24 is drawn between the wearer's legs and rests snugly against the wearer's body. When this embodiment is adjusted properly, the front fastening devices of the diaper 30A and 30B rest on the wearer's hips. In this embodiment, the shirt garment is crafted with short sleeves for the arms 83A and 83B; however, other embodiments could be sleeveless, have sleeves of varying lengths, have shoulder straps, among other styles. The wearer's head emerges through a neck hole 81.

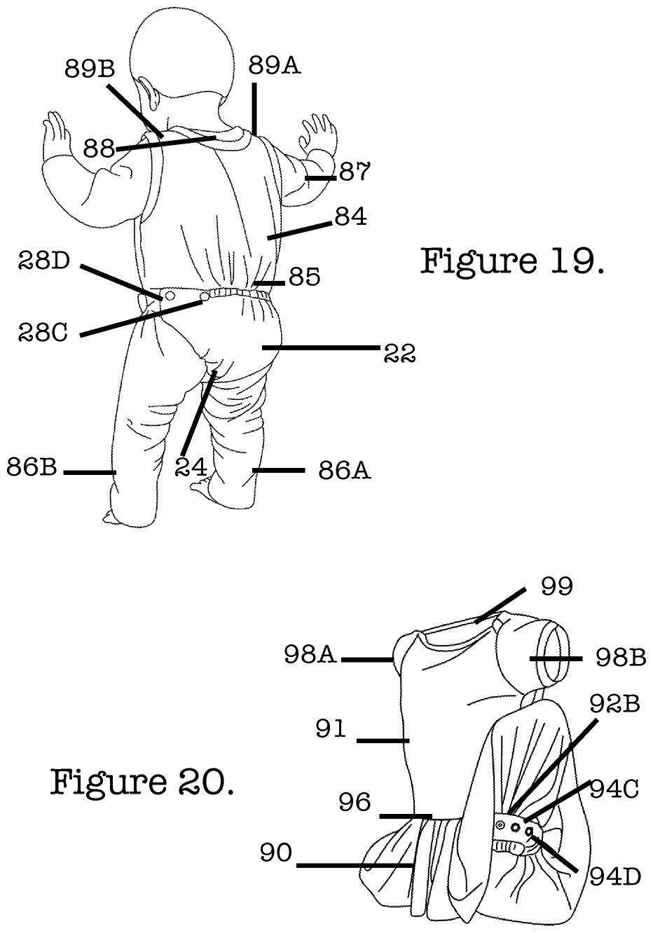

FIG. 19 illustrates a rear view of the diaper fully attached to a bodysuit garment with a waist belt 85, which is positioned on a wearer. The bodysuit garment has sleeves for the legs 86A and 86B as well as a shirt portion 84. This embodiment could be constructed by modifying the pattern for the pant garment shown in FIG. 17 by adding a shirt portion; fastening devices and elastic elements are then added in a similar fashion as described for the previously illustrated garments. Suitable materials for the bodysuit garment would be wool, fleece, or other fabrics used for clothing.

The wearer illustrated in FIG. 19 is wearing an undershirt 87 layered beneath the bodysuit garment, as the garment in this embodiment is sleeveless. Fastening devices 89A and 89B are added at the shoulders in this embodiment. These fastening devices could be snaps, hook and loop, buttons, among other materials. Instead of being sleeveless, other embodiments could have attached sleeves of varying lengths or shoulder straps with fastening devices located at the wearer's chest.

The entire circumference of the upper portion of the waist belt 85 is attached to the entire lower edge of the shirt portion 84. Furthermore, the waist belt 85 is attached at the hips to the sleeves for the legs 86A and 86B, leaving the wearer's groin and buttocks exposed in a similar fashion to the pant garment described above. FIG. 19 illustrates how the exposed groin and buttocks are covered by the diaper when it is fully attached to the bodysuit garment. The rear part of the diaper 22 hugs snugly to the rear part of the waist belt 85 of the bodysuit garment. The intermediate crotch part of the diaper 24 is drawn between the wearer's legs and rests snugly against the wearer's body. When this embodiment is adjusted properly, the rear fastening devices of the diaper, 28C and 28D being shown in FIG. 19, rest on the wearer's hips. This embodiment illustrates sleeves for the legs 86A and 86B that are cut long enough to cover the wearer's legs all the way to the heels of the wearer's feet; however, different lengths could be used for other embodiments, including embodiments with sleeves ending above the wearer's knees or with sleeves that have covers for the feet. The wearer's head emerges through a neck hole 88.

FIG. 20 illustrates a dress garment similar to the skirt garment described above. This embodiment could be constructed in a similar fashion as the skirt garment, with the additional step of crafting a shirt portion 91 that is attached to a waist belt 96 along with skirt material 90. Suitable materials for the shirt portion 91 and skirt material 90 would include cotton knit, polyester blends, fleece, wool, among others. This embodiment is illustrated with short sleeves for the arms 98A and 98B; however, other embodiments could be sleeveless, have sleeves of varying lengths, have shoulder straps, among other styles. A neck hole 99 is provided for the wearer's head to protrude through.

In FIG. 20, a portion of the skirt material 90 is shown as being lifted to reveal a front fastening device 92B, a male snap in this embodiment, and two rear fastening devices 94C and 94D, female snaps in this embodiment, located at the side of the waist belt 96. Although not pictured, a corresponding set of fastening devices are placed on the other side of the waist belt 96. When the skirt material 90 is allowed to hang freely, all of the fastening devices are concealed beneath the skirt material in this embodiment.

### Operation of Alternative Embodiments—FIGS. 1, 10, 13, 18, 19, 20

In operation, the pant garment, skirt garment, shirt garment, bodysuit garment, dress garment, or any other embodiment of garment can replace the waist belt described in the operation of the waist belt and diaper above. For the pant garment, the wearer's legs are fed into the waist of the pant garment shown in FIG. 10; then the legs are fed into the individual upper leg holes 58A and 58B. The feet are fed through the sleeves 62A and 62B and come out the lower foot holes 60A and 60B. When the garment is correctly on the wearer, the waist belt of the pant garment will rest on the wearer's hips in the same way as the waist belt alone described above. The rear part of the pant garment 54 will be aligned with the wearer's spine and the front part of the pant garment 52 will be aligned with the wearer's navel. The wearer's buttocks and groin will be exposed until the diaper is fastened to the pant garment. The diaper can then be fastened to the waist belt of the pant garment by fastening the front fastening devices 30A and 30B of the diaper shown in FIG. 1 to the front fastening devices of the pant garment 56A and 56B, respectively, followed by a choice of rear fastening devices as described above. Alternatively, the rear fastening devices may be fastened first followed by the front fastening devices, or the diaper can be fully or partially fastened to the pant garment prior to applying the pant garment to the wearer and then pulled on like a pull-up, among other methods of application. The caregiver can then enjoy changing the wearer's diaper and offering the toilet to the wearer in the same manner described above for the waist belt. Unless it is soiled with feces, the pant garment can be worn for many diaper changes before needing to be removed and washed.

In operation of the skirt garment, the wearer's legs are fed into the waist of the skirt garment shown in FIG. 13. When the garment is correctly on the wearer, the waist belt of the skirt garment will rest on the wearer's hips in the same way as the waist belt alone described above. The rear part of the skirt garment 70 will be aligned with the wearer's spine and the front part of the skirt garment 68 will be aligned with the wearer's navel. The diaper can then be fastened to the waist belt of the skirt garment underneath the skirt material 72 by fastening the front fastening devices 30A and 30B of the diaper to the front fastening devices of the skirt garment 78A and 78B, respectively, followed by a choice of rear fastening devices as described above. Alternatively, the rear fastening devices may be fastened first followed by the front fastening devices, or the diaper can be fully or partially fastened to the skirt garment prior to applying the skirt garment to the wearer and then pulled on like a pull-up, among other methods of application. The caregiver can then enjoy changing the wearer's diaper and offering the toilet to the wearer in the same manner described above for the waist belt. Unless it is soiled with feces, the skirt garment can be worn for many diaper changes before needing to be removed and washed.

In operation of the shirt garment shown in FIG. 18, the wearer's head, torso, and arms are first fed into the waist belt 82 of the shirt garment prior to attaching the diaper. Next, the wearer's arms are fed through the sleeves 83A and 83B and the wearer's head is fed through the neck hole 81. The waist belt 82 is pulled downward until it rests on the wearer's hips.

In operation of the bodysuit garment shown in FIG. 19, the fastening devices at the shoulders 89A and 89B are first unfastened, thereby enlarging the neck hole 88 so that the wearer's legs followed by the torso can be inserted into the garment. The wearer's legs then continue downward through the waist belt 85 until they are fed individually into the sleeves for the legs 86A and 86B. In this embodiment, the feet are exposed at the bottom of the sleeves 86A and 86B, however, foot coverings could also be included. The waist belt 85 then rests at the wearer's hips. The shoulder fastening devices 89A and 89B are then fastened to secure the garment over the wearer's shoulders. The wearer's groin and buttocks are exposed at this stage of the operation.

In operation of the dress garment shown in FIG. 20, the wearer's head, torso, and arms are first fed into the waist belt 96 of the dress garment. Next, the wearer's arms are fed through the sleeves 98A and 98B and the wearer's head is fed through the neck hole 99. The waist belt 96 is pulled downward until it rests on the wearer's hips with the skirt material 90 hanging freely.

For each of the garments shown in FIGS. 18, 19, and 20 described above, the diaper can then be fastened to the waist belt of the garment by fastening the front fastening devices 30A and 30B of the diaper to front fastening devices of the garment followed by a choice of rear fastening devices as described above. Alternatively, the rear fastening devices may be fastened first followed by the front fastening devices, among other methods of application. The caregiver can then enjoy changing the wearer's diaper and offering the toilet to the wearer in the same manner described above for the waist belt. Unless they are soiled with feces, the shirt garment, bodysuit garment, and dress garment can all be worn for many diaper changes before needing to be removed and washed.

### Advantages

The above-described embodiment provides an advantage in that the absorbent article is compatible with many varieties of garments such as the waist belt, the pant garment, the skirt garment, the shirt garment, the bodysuit garment, and the dress garment described above, among other variations. Thus, the caregiver has the flexibility to choose between several different garments while still using the same diapers. The waist belt shown in FIG. 3 is a good choice for hot climates, hot seasons, for layering under traditional clothes or bodysuits, among other situations. The longer pant garment shown in FIG. 12 is a good choice for cold seasons, cold climates, among other situations. The shorter pant garment shown in FIG. 11 is a good choice for warm climates, warm seasons, for stylistic reasons, among other situations. The skirt garment shown in FIG. 15 is also a good stylistic choice for many different occasions or reasons.

Before an infant learns to walk, shirts and dresses that hang freely and do not snap or fasten beneath the groin tend to ride up the infant's torso while the infant is crawling, rolling, or being held by a caregiver. Traditional bodysuits, rompers, overalls, and other similar garments that fasten below the groin prevent the infant's navel and back from being exposed and are therefore a popular choice among caregivers. The embodiments illustrated in FIGS. 18, 19, and 20 all retain this advantage in addition to many of the other advantages discussed herein. The shirt garment shown in FIG. 18, for example, would be a good choice for keeping the wearer's torso covered in hot or warm climates or for layering under traditional pants, among other situations. The bodysuit garment shown in FIG. 19 would be a good choice for keeping the wearer's torso covered in cold seasons, cold climates, for bedtime or naps, among other situations. The dress garment shown in FIG. 20 would be a good stylistic choice for keeping the wearer's torso covered for many different occasions or reasons.

In this embodiment, the diaper, waist belt, pant garment, skirt garment, shirt garment, bodysuit garment, dress garment, and other variations of garment are all washable, reusable, adjustable, and compatible. Therefore, a caregiver could conceivably purchase a desired number of diapers along with a choice of waist belts, pant garments, skirt garments, shirt garments, bodysuit garments, dress garments, or other compatible garments and have all the equipment needed to enjoy the following benefits:
1. The caregiver will have enough diapers to last a significant portion of multiple wearers' diaper-wearing years, as the embodiment is washable, reusable, and can be tightened or loosened as the wearer grows.
2. The caregiver can quickly and easily attach the wearer's diaper without restricting the wearer's position, regardless of the garment of choice.
3. The caregiver can quickly and easily detach the wearer's diaper without removing the compatible garment and without restricting the wearer's position, regardless of the garment of choice.
4. The caregiver can quickly and easily change the wearer's diaper without removing the compatible garment and without restricting the wearer's position, regardless of the garment of choice.
5. The caregiver can offer the toilet or other receptacle to the wearer in a quick and efficient manner without removing the diaper or compatible garment from the wearer, regardless of the garment of choice.
6. The caregiver has a choice of opening the diaper from the front or the rear when offering the toilet or other receptacle to the wearer, regardless of the garment of choice.
7. The caregiver can tend to the wearer's needs in a variety of situations that are problematic for traditional diapers, such as in restrooms without changing pads or in the situation that the caregiver is disabled, regardless of the garment of choice.
8. The wearer can enjoy the independence of being able to put on and change the diaper without the aid of a caregiver.
9. The embodiment uses snaps instead of hook and loop closures, which are sturdier and hold up better to repeated washing.
10. The embodiment uses four fastening devices to successfully attach the diaper to the compatible garment, regardless of the garment of choice.
11. The embodiment can be applied to diaper-wearers of all descriptions, including infants, children, and adult incontinent individuals.
12. The embodiment's absorbency can be easily adjusted to suit the caregiver's and wearer's needs.
13. The caregiver can choose a compatible garment that will keep the wearer's torso covered while still enjoying the benefits listed above.
14. The benefits listed above will not be compromised by climate, season, or stylistic choices, as the diapers are compatible with multiple types of garments suitable for multiple situations and weather conditions.

### CONCLUSIONS, RAMIFICATION, AND SCOPE

While the above description contains many specificities, these should not be construed as limitations on the scope, but rather as an exemplification of several embodiments thereof. Many other variations are possible. For example, a garment with sleeves for the legs as well as a skirt would be one possible variation. Different lengths for the skirt are conceivable. Other types of garments with upper body coverings such as overalls or rompers could also be adapted to be compatible with the diapers shown in this embodiment. For embodiments with upper body coverings, many numbers of variations on the length and style for the arm sleeves are conceivable. A waist belt without belt closure devices that is always in the closed position of FIG. 4 is also conceivable. Both the diapers and the garments could be made in a variety of colors or patterns from a variety of materials. The diaper could also be made without a liquid impervious top sheet, transforming the diaper into a training pant or padded underwear; this would be an advantage to wearers who are transitioning from diapers to underpants. It is also conceivable to create disposable diapers that would be compatible with disposable or reusable belts and garments. Accordingly, the scope of the disclosure should be determined not by the embodiments illustrated, but by the appended claims and their legal equivalent.

I claim:

1. A garment comprising:
an absorbent article having a longitudinal direction and a lateral direction and comprising of a front part, a rear part, and an intermediate crotch part
a plurality of compatible secondary articles, said secondary articles comprising of a waist belt with a front part and a rear part
a plurality of front fastening means on said front part of said absorbent article and on said front parts of said waist belts of said compatible secondary articles, said front fastening means thereby enabling said front part of said absorbent article to be attached to said front part of said waist belt of any chosen one of said secondary articles, hereby referred to as the garment of choice
a plurality of rear fastening means on said rear part of said absorbent article and on said rear parts of said waist belts of said compatible secondary articles, said rear fastening means thereby enabling said rear part of said absorbent article to be attached to said rear part of said waist belt of said garment of choice
whereby said garment of choice can be worn by a wearer and said front fastening means and said rear fastening means can be employed in any order to attach said absorbent article to said garment of choice without restricting said wearer's position such that said absorbent article fits snugly against said wearer to contain bodily excretions and said front fastening means and said rear fastening means can be released in any order to fully or partially detach said absorbent article from said garment of choice without restricting said wearer's position or removing said garment of choice from said wearer.

2. The garment of choice of claim 1, further including leg-covering means in the form of two attached sleeves of material of predetermined length positioned on each side of said waist belt at the join of said front part and said rear part so that said wearer's legs may be inserted into said sleeves of material thereby fully or partially covering said legs by said sleeves of material when said garment of choice is worn by said wearer.

3. The garment of choice of claim 1, further including a predetermined length of material along one lateral edge of said waist belt which hangs over said wearer's hips in the form of a skirt.

4. The garment of choice of claim 3, further including leg-covering means in the form of two attached sleeves of material of predetermined length positioned on each side of said waist belt at the join of said front part and said rear part so that said wearer's legs may be inserted into said sleeves of material thereby fully or partially covering said legs by said sleeves of material when said garment of choice is worn by said wearer.

5. The garment of choice of claim 4, further including a belt fastening means thereby allowing said waist belt to be opened and closed around the waist of said wearer.

6. The garment of choice of claim 3, further including a belt fastening means thereby allowing said waist belt to be opened and closed around the waist of said wearer.

7. The garment of claim 1 wherein said front fastening means and said rear fastening means are made of snaps.

8. The rear fastening means of claim 7, further including size adjustment means in the form of a plurality of male snaps, alternatively female snaps, on the lateral edges of said rear part of said absorbent article and a plurality of female snaps, alternatively male snaps, on said rear parts of said waist belts of said compatible secondary articles, thereby allowing said absorbent article to be loosened or tightened against said waist belt of said garment of choice according to the size of said wearer by fastening a choice of said male snaps to a choice of said female snaps in a range of combinations.

9. The absorbent article of claim 1, further including elastic means in said rear part and on the lateral sides of said intermediate crotch part thereby providing a form-fitting shape.

10. The absorbent article of claim 1, further including liquid containment means in the form of a liquid impervious top sheet fully covering said front part, said rear part, and said intermediate crotch part, thereby containing moisture within said absorbent article.

11. The absorbent article of claim 10, further including increased absorption means in the form of an inner facing material of the same predetermined shape as said liquid impervious top sheet and which is attached to said liquid impervious top sheet along the outermost lateral and front edges of said inner facing material and said liquid impervious top sheet, thereby forming a pocket with an opening in said rear part of said absorbent article into which additional absorbent material can be inserted to increase said absorbent article's absorbency.

12. The absorbent article of claim 10 wherein said liquid impervious top sheet is made of polyurethane laminate fabric.

13. The compatible secondary articles of claim 1, further including elastic means in said rear part of said waist belt thereby providing a form-fitting shape.

14. The garment of choice of claim 1, further including a belt fastening means thereby allowing said waist belt to be opened and closed around the waist of said wearer.

15. The garment of choice of claim 14, further including leg-covering means in the form of two attached sleeves of material of predetermined length positioned on each side of said waist belt at the join of said front part and said rear part so that said wearer's legs may be inserted into said sleeves of material thereby fully or partially covering said legs by said sleeves of material when said garment of choice is worn by said wearer.

16. The garment of choice of claim 1, further including torso-covering means in the form of an upper body garment which is fully or partially attached to said waist belt of said garment of choice thereby covering said wearer's upper body when said garment of choice is worn by said wearer.

17. The garment of choice of claim 16, further including leg-covering means in the form of two attached sleeves of material of predetermined length positioned on each side of said waist belt at the join of said front part and said rear part so that said wearer's legs may be inserted into said sleeves of material thereby fully or partially covering said legs by said sleeves of material when said garment of choice is worn by said wearer.

18. The garment of choice of claim 17, further including a predetermined length of material along one lateral edge of said waist belt which hangs over said wearer's hips in the form of a skirt.

19. The garment of choice of claim 16, further including a predetermined length of material along one lateral edge of said waist belt which hangs over said wearer's hips in the form of a skirt.
